(19) United States
(12) Patent Application Publication
(10) Pub. No.: US 2023/0016479 A1
(43) Pub. Date: Jan. 19, 2023

(54) METHOD FOR PREPARING MESENCHYMAL STEM CELLS HAVING IMPROVED VIABILITY THROUGH ANTI-CANCER VIRUS INTRODUCTION

(71) Applicants: Jeong Chan RA, Chungcheongbuk-do (KR); R Bio CO., Ltd., Seoul (KR); NATURECELL CO., LTD., Seoul (KR)

(72) Inventors: Jeong Chan RA, Chungcheongbuk-do (KR); Eun-Young KIM, Seoul (KR)

(21) Appl. No.: 17/784,740

(22) PCT Filed: Dec. 14, 2020
(86) PCT No.: PCT/KR2020/018272

§ 371 Date: Jun. 13, 2022

(30) Foreign Application Priority Data
Dec. 13, 2019 (KR) 10-2019-0166517

(51) Int. Cl. C12N 5/0775; A61K 35/28; A61P 35/00

(52) U.S. Cl. CPC C12N 5/0667; A61K 35/28; A61P 35/00; C12N 2500/38; C12N 2760/18432; C12N 2501/999

(57) ABSTRACT

The present invention relates to a method for preparing oncolytic virus-containing mesenchymal stem cells having improved cell viability, a method for storing the oncolytic virus-containing stem cells produced by the method, and a cell therapeutic agent for cancer treatment containing the oncolytic virus-containing stem cells produced by the method. More particularly, an oncolytic virus is introduced into mesenchymal stem cells, followed by treatment with aspirin, so that the infection efficiency of the oncolytic virus may be increased, the replication time of the virus may be prolonged, and lysis of the stem cells by the virus may be prevented, thereby improving the viability and survival period of the stem cells and preparing anticancer stem cells having excellent activity. The anticancer stem cell therapeutic agent produced in this way is maintained at high viability during cold storage due to aspirin treatment, and thus is very useful medically and industrially.

### TECHNICAL FIELD

The present invention relates to a method for preparing oncolytic virus-containing mesenchymal stem cells having improved cell viability, a method for storing the mesenchymal stem cells, and a cell therapeutic agent for cancer treatment containing the stem cells produced by the method, and more particularly, to the production of an anticancer stem cell therapeutic agent having excellent activity, in which an oncolytic virus is introduced into mesenchymal stem cells, followed by treatment with aspirin, so that the replication time of the oncolytic virus may be prolonged and lysis of the stem cells by the virus may be prevented, thereby improving the viability and survival period of the mesenchymal stem cells.

### BACKGROUND ART

The term “stem cells” refers to cells that can self-renew and differentiate into two or more cell types. Stem cells may be classified into totipotent stem cells, pluripotent stem cells, and multipotent stem cells.

Totipotent stem cells are cells having totipotent properties capable of developing into one perfect individual, and these properties are possessed by cells up to the 8-cell stage after fertilization of an oocyte by a sperm. When these cells are isolated and transplanted into the uterus, they can develop into one perfect individual.

Pluripotent stem cells, which are cells capable of developing into various cells and tissues derived from the ectodermal, mesodermal and endodermal layers, are derived from an inner cell mass located inside of blastocysts generated 4 to 5 days after fertilization. These cells are also called embryonic stem cells and can differentiate into various other tissue cells but cannot form new living organisms.

Multipotent stem cells are stem cells that can differentiate only into cells specific to the tissue and organ containing these cells, and are involved not only in the growth and development of each tissue and organ in the fetal, neonatal and adult stages, but also in maintaining homeostasis of adult tissues and inducing regeneration in the event of tissue damage, and tissue-specific multipotent are collectively referred to as mesenchymal stem cells.

Mesenchymal stem cells (Rebecca S Y Wong, et al., J Biomed Biotechnol 24:2011, 2011) have been used for cell-based therapy in a variety of disease conditions, such as heart disease, osteogenesis imperfecta and spinal cord injury, and the results thereof have attracted attention.

Meanwhile, “cancer” is characterized by “uncontrolled cell growth”, and a cell mass called a tumor is formed by this abnormal cell growth, invades the surrounding tissue, and also metastasizes to other organs of the body in severe cases. Academically, cancer is also called neoplasia.

Methods for treating cancer include surgery, radiotherapy, and chemotherapy, i.e., administration of anticancer drugs, and currently, many studies are being conducted around the world to develop cancer immunotherapy. Many studies have been conducted to conquer cancer by using various platforms such as bi-specific T-cell engagers (BiTEs) from immune checkpoint inhibitors, CAR-T (chimeric antigen receptor T-cells or NK cells), and oncolytic viruses.

In particular, the biggest attraction of oncolytic viruses, claimed by experts who study oncolytic viruses, is that the viruses are alive, unlike existing treatments. In addition, viruses are attractive in that they can reproduce by themselves because they possess their own genes, and thus they can infect and destroy nearby cancer cells in addition to the injection site. Of course, in order for the oncolytic viruses to be commercially used in clinical practice, it is necessary to overcome a big hurdle, that is, overcoming the stereotype that self-propagating viruses cause various diseases. Nevertheless, there is a great need for the development of oncolytic virus technology for the purpose of cure rather than simply prolonging life.

In addition, the side effects of existing cancer treatment methods during treatment have been a major obstacle in the field of cancer treatment, and the existing cancer treatment methods cannot be free from the risk of recurrence after treatment. In addition, there is a need for alternatives to prevent cancer in environments that cannot be free from cancer, for example, people who have a family history of cancer or have been immunocompromised due to various diseases.

Accordingly, the present inventors have made extensive efforts to develop a cancer therapy product using oncolytic virus-containing stem cells having excellent anticancer efficacy while having few side effects due to their specificity to cancer, and as a result, have found that, when an oncolytic virus is introduced into mesenchymal stem cells, followed by treatment with aspirin, the replication time of the oncolytic virus may be prolonged and lysis of the stem cells by the virus may be prevented, thereby improving the viability and survival period of the stem cells and preparing an anticancer stem cell therapeutic agent which has excellent activity, thereby completing the present invention.

### SUMMARY OF THE INVENTION

An object of the present invention is to provide a method for preparing mesenchymal stem cells containing an oncolytic virus, in which mesenchymal stem cells into which the oncolytic virus has been introduced are treated with aspirin, so that the replication time of the oncolytic virus may be prolonged and lysis of the stem cells by the virus may be prevented, thereby improving the viability and survival period of the stem cells, a method for storing the mesenchymal stem cells produced by the above method, and a cell therapeutic agent for cancer treatment containing, as an active ingredient, mesenchymal stem cells produced by the above method.

To achieve the above object, the present invention provides a method for preparing oncolytic virus-containing mesenchymal stem cells having improved cell viability, the method comprising steps of: (a) preparing pelleted mesenchymal stem cells; (b) infecting the pelleted mesenchymal stem cells with an oncolytic virus; and (c) separating the mesenchymal stem cells infected with the oncolytic virus to obtain mesenchymal stem cells into which the oncolytic virus has been introduced and which have improved cell viability.

The method also provides a method of storing the mesenchymal stem cells, produced by the above method, using an autologous serum containing aspirin.

The present invention also provides a cell therapeutic agent for cancer treatment containing, as an active ingredient, the mesenchymal stem cells produced by the above method.

The present invention also provides a method for preventing or treating cancer comprising a step of administering the mesenchymal stem cells produced by the above method.

The present invention also provides the use of the mesenchymal stem cells, produced by the above method, for the prevention or treatment of cancer.

The present invention also provides the use of the mesenchymal stem cells, produced by the above method, for the production of a cell therapeutic agent for the prevention or treatment of cancer.

### BRIEF DESCRIPTION OF DRAWINGS

FIG. 1a depicts fluorescence micrographs showing the results of identifying MVeGFP by FITC fluorescence after infecting adipose-derived stem cells with measles virus.

FIG. 1b is an electron micrograph showing adipose-derived stem cells after infection with measles virus (arrow: measles virus in stem cells).

FIG. 2 shows the results of analyzing the expression of CD46, CD150 and nectin-4 in cancer cell lines by FACS.

FIG. 3 shows the results of analyzing the killing ability of MVeGFP in cancer cell lines and stem cells by cytopathic effect (CPE) assay.

FIG. 4 shows the results of analyzing the killing ability of MVeGFP at 1000 TCID50/ml in breast cancer cell lines by CPE assay.

FIG. 5 shows the results obtained by infecting the MCF7 breast cancer cell line with MVeGFP and quantifying the extent of infection by FACS.

FIG. 6 shows the results of analyzing the degree of measles virus infection in flask-contained cells and pelleted cells by CPE assay.

FIG. 7 shows the results of CPE assay of pelleted cells infected with measles virus at different concentrations for different times.

FIG. 8a shows the results of analyzing the viability of non-cultured ViroSTEM after infection with measles virus.

FIG. 8b shows the results of analyzing the viability of ViroSTEM cultured for 4 days after infection with measles virus.

FIG. 9a shows the results of analyzing the viability of ViroSTEM cold-stored in 30% autologous serum-containing physiological saline treated with different concentrations of aspirin.

FIG. 9b shows the results of comparing the viability of ViroSTEM, cold-stored for 3 days in 30% autologous serum-containing physiological saline treated with different concentrations of aspirin, with that of each control.

FIG. 10 shows the results obtained by cold-storing ViroStem in autologous serum containing aspirin for up to 80 hours and then analyzing the viability of ViroStem and comparing the viability with that of each control.

FIG. 11 shows the results of evaluating the expiration date of ViroSTEM by qPCR.

FIG. 12 the results of evaluating the anticancer effect of ViroSTEM treated with aspirin against a metastatic breast cancer cell line (MDA-MB-231).

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

In the present invention, an oncolytic virus was introduced into mesenchymal stem cells, followed by treatment with aspirin, so that the replication time of the oncolytic virus could be prolonged and the lysis of the stem cells by the virus could be prevented, thereby improving the viability and survival period of the mesenchymal stem cells infected with the oncolytic virus and preparing a stem cell therapeutic agent having excellent activity and an increased cold storage period. Accordingly, it was confirmed that the period during which the measles virus-infected stem cells produced by the method of the present invention showed a viability of 80% in a cold state increased up to 80 hours.

Therefore, in one aspect, the present invention is directed to a method for preparing oncolytic virus-containing mesenchymal stem cells having improved cell viability, the method comprising steps of: (a) preparing pelleted mesenchymal stem cells; (b) infecting the pelleted mesenchymal stem cells with an oncolytic virus; and (c) separating the mesenchymal stem cells infected with the oncolytic virus to obtain mesenchymal stem cells into which the oncolytic virus has been introduced and which have improved cell viability.

In the present invention, the mesenchymal stem cells in step (a) are preferably cultured in a medium containing aspirin, and the concentration of the aspirin is preferably 0.1 mM to 1 mM, without being limited thereto.

In the present invention, the medium containing aspirin preferably further contains vitamin C, without being limited thereto.

In the present invention, the medium is preferably DMEM or K-SFM containing 5 to 10% FBS and N-acetyl cysteine (NAC) and more preferably further contains calcium, rEGF, insulin and hydrocortisone, without being limited thereto.

In the present invention, the mesenchymal stem cells in step (a) are preferably pretreated with vitamin C, without being limited thereto.

In one example of the present invention, adipose-derived mesenchymal stem cells cultured in a vitamin C-containing medium were cultured in a medium containing aspirin, thereby preparing mesenchymal stem cells having improved ability to inhibit cancer cell proliferation. That is, the produced mesenchymal stem cells may be: anticancer stem cells produced by culturing adipose-derived mesenchymal stem cells, pretreated with vitamin C and cultured, in a medium containing aspirin; or anticancer stem cells produced by adipose-derived mesenchymal stem cells in a medium containing both vitamin C and aspirin; or anticancer stem cells produced by culturing adipose-derived mesenchymal stem cells, pretreated with vitamin C and cultured, in a medium containing vitamin C and aspirin. The present inventors named all these cells as “Angel-Stem Cells” (WO/2018/021879).

In one example of the present invention, the extents of oncolytic virus infection of pelleted stem cells flask-contained stem cells were analyzed, and it was confirmed that the extent of measles virus infection of the pelleted stem cells was better.

In particular, in order to increase the extent of infection, it is preferable to centrifuge measles virus-infected stem cells, thereby applying physical changes thereto.

In the present invention, the mesenchymal stem cells in step (b) are preferably 1×10⁵ to 1×10⁶ cells, more preferably 1×10⁵ to 3×10⁵ cells, most preferably 2×10⁵ cells, without being limited thereto.

In the present invention, the mesenchymal stem cells may be derived from a tissue selected from the group consisting of adipose, uterus, bone marrow, muscle, placenta, umbilical cord blood, urine, hair follicle, and skin tissues.

As used herein, the term “stem cells” refers to cells that can self-renew and differentiate into two or more cell types, and the term “adult stem cells” refers to stem cells that appear either in the stage in which each organ of an embryo is formed after the developmental process or in the adult stage.

As used herein, the term “mesenchymal stem cells” refers to undifferentiated stem cells isolated from human or mammalian tissue and may be derived from various tissues. Particularly, the mesenchymal stem cells may be umbilical cord-derived mesenchymal stem cells, umbilical cord blood-derived mesenchymal stem cells, bone marrow-derived mesenchymal stem cells, adipose-derived mesenchymal stem cells, muscle-derived mesenchymal stem cells, nerve-derived mesenchymal stem cells, skin-derived mesenchymal stem cells, amnion-derived mesenchymal stem cells, or placenta-derived mesenchymal stem cells. Technology for isolating stem cells from each tissue is already known in the art.

As used herein, the term “adipose tissue-derived mesenchymal stem cells” refers to undifferentiated adult stem cells isolated from adipose tissue. A method for isolating adipose tissue-derived mesenchymal stem cells may be, for example, as follows. That is, adipose-derived mesenchymal stem cells may be isolated by a method of culturing an adipose-containing suspension (in physiological saline) obtained by liposuction, and then either collecting a stem cell layer, attached to a culture vessel such as a flask, by trypsin treatment, or directly collecting stem cells suspended in a small amount of physiological saline by rubbing with a scraper.

These “adipose tissue-derived adult stem cells” or “adipose tissue-derived mesenchymal stem cells” are undifferentiated adult stem cells isolated from adipose tissue, and are also referred herein as “adipose stem cells” for short. These cells may be obtained through a conventional method known in the art.

As a medium that is used for obtaining the adipose stem cells, a conventional medium known in the art to be suitable for stem cell culture may be used. Preferably, DMEM (Dulbecco's modified Eagle medium) or keratinocyte-SFM (keratinocyte serum free medium) may be used, and a mixed medium of IMDM (Iscove's Modified Dulbecco's Medium), a-MEM (Alpha Modification of Eagle's Medium), F12 (Nutrient Mixture F-12) and DMEM/F12 (Dulbecco's Modified Eagle Medium: Nutrient Mixture F-12) may also be used without being limited thereto.

The medium for culturing adipose stem cells may be supplemented with additives which promote proliferation of an undifferentiated phenotype of mesenchymal stem cells while inhibiting differentiation of the stem cells. In addition, the medium may contain a neutral buffer (for example, phosphate and/or high concentration bicarbonate) in isotonic solution, and protein nutrients (for example, serum such as fetal bovine serum (FBS), serum replacements, albumin, or essential or non-essential amino acids such as glutamine). Furthermore, the medium may contain lipids (fatty acids, cholesterol, an HDL or LDL extract of serum) and other components found in most preservation media of this kind (such as insulin or transferrin, nucleosides or nucleotides, pyruvate, a sugar source such as glucose, selenium in any ionized form or salt, a glucocorticoid such as hydrocortisone and/or a reducing agent such as β-mercaptoethanol).

In addition, it may be beneficial that the medium contains anti-clumping agents, for example, those sold by Invitrogen (Cat #00100571AE) in order to prevent cells from adhering to each other, adhering to a vessel wall, or forming excessively large clusters.

In particular, the medium for obtaining or culturing adipose stem cells, which is used in one embodiment of the present invention, preferably contains: a basal medium selected from the group consisting of DMEM, defined keratinocyte-SFM, α-MEM, IMDM, F12 and DMEM/F12; a medium composition for culturing mesenchymal stem cells containing L-ascorbic acid 2-phosphate (vitamin C), fetal bovine serum and N-acetyl-L-cysteine; and aspirin, but the present invention is not limited thereto.

In the present invention, the medium may contain, but is not limited to, 0.05 to 1 mM ascorbic acid 2-phosphate, 2 to 20% fetal bovine serum, 0.2 to 20 mM N-acetyl-L-cysteine and 0.1 to 1 mM aspirin.

In the present invention, the oncolytic virus is preferably measles virus, more preferably MV (Edmonston strain) virus, without being limited thereto.

Measles virus is known to have anticancer efficacy against malignant tumors (Msaouel P et al., Curr Pharm Biotechnol. 13(9):1732-41, 2012), CSC (cancer stem cell) (Fang Huang et al., World J Gastroenterol. 22(35):7999-8009, 2016), lung cancer (Zhao D et al., Oncol Rep. 29(1):199-204, 2013; Ong H T et al., J Hepatol. 59(5):999-1006, 2013), blood tumors (D Grote et al., Blood. 97(12):3746-54, 2001), ovarian cancer (Zhou S et al., Cancer Lett. 318(1):14-25, 2012), myeloma (Peng K W, et al. Blood. 98(7):2002-2007, 2001), breast cancer (McDonald C J et al., Breast Cancer Res Treat. 99(2):177-84, 2006), brain cancer (Phuong L K, et al., Cancer Res. 63(10):2462-2469, 2003), colorectal adenocarcinoma (Nicolas Boisgerault et al., Biomed Res Int. 11, 2013), and osteosarcoma (Evidio Domingo Musibay et al., Cancer Gene Ther. 21(11): 483-490, 2014). It is believed that, because stem cells carrying this oncolytic virus are specific to cancer, they may induce the effect of increasing anticancer efficacy while having reduced side effects, and can also play a role in the ultimate goal of prolonging healthy lifespan.

In the present invention, the oncolytic virus in step (b) may be attenuated measles vaccine virus. As the attenuated virus, a commercially available virus may be used, or a wild-type virus or a commercially available attenuated virus may also be used after attenuation and additional attenuation.

In one example of the present invention, the GFP-tagged measles virus MVeGFP was used.

In the present invention, the oncolytic virus in step (b) preferably has 1×10⁵ to 1×10⁷ TCID50, more preferably 1×10⁵ to 5×10⁶ TCID50, most preferably 1×10⁶ TCID50, without being limited thereto.

In the present invention, the infection in step (b) is preferably performed at 36 to 37° C. for 30 minutes to 2 hours, more preferably 30 minutes, without being limited thereto.

In the present invention, the oncolytic virus-infected mesenchymal stem cells of step (c) are preferably further treated with aspirin, and are more preferably treated with aspirin added to a vehicle containing autologous serum, without being limited thereto.

In one example of the present invention, the mesenchymal stem cells were treated with aspirin at a concentration of 10 μg/ml to 100 μg/ml, and the most effective concentration of aspirin was 50 μg/ml. The treatment with aspirin was performed in a state in which the aspirin was added to a vehicle containing autologous serum.

The content of the autologous serum is preferably 10% to 50%, more preferably 30%, without being limited thereto.

In addition, the vehicle is preferably at least one selected from the group consisting of physiological saline, Hartmann-D solution, and PBS. In one example of the present invention, physiological saline was used.

In the present invention, the treatment with aspirin is preferably performed at a cold temperature, more preferably at 0.1 to 10° C., most preferably at 4° C., without being limited thereto.

In general, when mesenchymal stem cells are infected with measles virus at an MOI of 1.0, the period of time during which the viability of the virus-infected mesenchymal stem cells is 80% or more is only 48 hours (Mader E K et al., Clin Cancer Res. 1;15(23):7246-55, 2009). However, when the measles virus-infected mesenchymal stem cells of the present invention were treated with aspirin, the period of time during which the viability of the mesenchymal stem cells was 80% or more increased to 80 hours or more.

In one example of the present invention, the cell viability of “ViroSTEM”, which is mesenchymal stem cells into which the oncolytic virus has been introduced, was analyzed immediately after infection and after culture. As a result, it was confirmed that the cell viability of ViroSTEM was better when it was not further cultured.

Thus, in the present invention, the mesenchymal stem cells in step (c) are preferably not further cultured after the oncolytic virus has been introduced thereinto, but the present invention is not limited thereto.

“Oncolytic virus-containing mesenchymal stem cells” produced by the method of the present invention may not only be stored but also treated with a composition containing aspirin and autologous serum, and have excellent anticancer activity for up to 80 hours. Therefore, the cell therapeutic agent produced in this way may be administered to a patient directly without additional culture or other additional manipulation.

In another aspect, the method is directed to a method of storing the mesenchymal stem cells, produced by the above method, using an autologous serum containing aspirin.

In the present invention, the autologous serum containing aspirin preferably further contains a vehicle, without being limited thereto.

The vehicle is preferably at least one selected from the group consisting of physiological saline, Hartmann-D solution and PBS. In one example of the present invention, physiological saline was used.

In the present invention, the concentration of the aspirin is preferably 10 μg/ml to 100 μg/ml, more preferably 50 μg/ml, without being limited thereto.

In the present invention, the content of the autologous serum is preferably 10% to 50%, more preferably 30%, without being limited thereto.

In the present invention, the treatment with aspirin is preferably performed at a cold temperature, more preferably 0.1 to 10° C., most preferably at 4° C., without being limited thereto.

In another aspect, the present invention is directed to a cell therapeutic agent for cancer treatment containing, as an active ingredient, the mesenchymal stem cells produced by the above method.

In general, when mesenchymal stem cells are infected with measles virus at an MOI of 1.0, the period of time during which the viability of the virus-infected mesenchymal stem cells is 80% or more is only 48 hours (Mader E K et al., Clin Cancer Res. 1;15(23):7246-55, 2009). In other words, since the virus lyses the stem cells, the survival period of the virus-infected stem cells is short. For this reason, it is impossible to commercialize stem cells infected with the oncolytic virus as a cell therapeutic agent.

However, when the measles virus-infected mesenchymal stem cells of the present invention were treated with aspirin, the period of time during which the viability of the mesenchymal stem cells was 80% or more increased to 80 hours or more. Thus, the anticancer stem cell therapeutic agent produced by the method of the present invention may be administered in an actual product form.

In particular, the anticancer stem cell therapeutic agent of the present invention may be commercialized without further culturing after infecting the pelleted stem cells with the oncolytic virus, followed by centrifugation. That is, the anticancer stem cell therapeutic agent may not only be stored but also treated with a composition containing aspirin and autologous serum and has excellent anticancer activity for up to 80 hours, and thus the cell therapeutic agent produced in this way may be administered to a patient directly without additional culturing or other additional manipulation.

In the present invention, the cancer is preferably lung cancer, hematological tumor, ovarian cancer, myeloma, breast cancer, brain cancer, rectal cancer, colon cancer, colorectal adenocarcinoma, osteosarcoma, or cancer stem cells, without being limited thereto.

In one example of the present invention, it was confirmed that the anticancer effect of the oncolytic virus was excellent on breast cancer cells and was the same on both an estrogen-dependent breast cancer cell line (MCF-7) and an estrogen-independent breast cancer cell line (MDA-MB-231).

In the present invention, the mesenchymal stem cells may be derived from a tissue selected from the group consisting of adipose, uterus, bone marrow, muscle, placenta, umbilical cord blood, urine, hair follicle, and skin tissues.

“Cancer” is characterized by “uncontrolled cell growth”, and a cell mass called a tumor is formed by this abnormal cell growth, invades the surrounding tissue, and also metastasizes to other organs of the body in severe cases.

The term “anticancer” is interpreted to include not only treating cancer diseases, that is, inhibiting the proliferation of cancer cells or cancer stem cells or killing cancer cells or cancer stem cells, but also preventing cancer diseases, that is, increasing resistance to cancer before the onset of cancer. Accordingly, in the present specification, the term “prevention or treatment of cancer” or “inhibition of cancer proliferation” is used interchangeably with the term “anticancer”.

In the present invention, the term “cancer cells” includes cells that undergo abnormal cell growth due to genetic variation (mutation) in proliferation and growth mechanisms of normal cells and have aggressive mobility to other organs, which may be referred to as “metastasis”. In addition, “cancer stem cells” are known to be present in tumors and are considered to be caused by abnormal transfer of genetic information from normal stem cells. Cancer stem cells are maintained and proliferate due to the presence of the microenvironment called “niche” for survival thereof, and the surrounding normal cells, immune-related cells or differentiated cancer cells are known to affect the maintenance of characteristics and proliferation of these cancer stem cells.

As used herein, the term “cell therapy injection product” or “cell therapeutic agent” refers to a pharmaceutical composition that contains stem cells for treating a tissue defect and is capable of correcting the defect by being administered parenterally, that is, being injected into the defect site or the site adjacent thereto in the form of injection.

The term “treating” means, unless otherwise stated, inhibiting the progress of, or reversing, alleviating or preventing either the disease or disorder to which the term applies, or one or more symptoms of such disease or disorder.

As used herein, the term “treatment” refers to an action of treatment when the term “treating” is defined as above.

Thus, the “treatment” or “therapy” of cancer in mammals includes one or more of the following:

(1) inhibiting growth of cancer, i.e., suppressing development thereof,

(2) preventing the diffusion of cancer, i.e., preventing metastasis,

(3) alleviating cancer, i.e., causing regression of cancer,

(4) preventing recurrence of cancer, and

(5) palliating symptoms of cancer.

Cancer is an intractable chronic disease that cannot be fundamentally cured in many cases even by treatment with surgery, radiotherapy and chemotherapy, causes pain to patients and ultimately leads to death. Over the past several decades, surgery, chemotherapy (treatment with anticancer drugs), radiotherapy and the like have been greatly advanced, but failed to provide the ultimate solution to cancer.

In the present invention, the oncolytic virus may be safely and effectively introduced into mesenchymal stem cells by controlling the infection environment, time and concentration, the incubation time of the virus may be prolonged, and lysis of the stem cells by the virus may be prevented, thereby improving the viability and survival period of the mesenchymal stem cells and preparing anticancer stem cells having enhanced anticancer efficacy. The anticancer stem cell therapeutic agent produced in this way has the advantage of maintaining a remarkably high viability during cold storage using aspirin.

In addition, as stem cells are infected with attenuated measles virus and treated with aspirin, the replication time of the virus is prolonged and the lysis of the stem cells by the virus is suppressed. Thus, the period of time during which the measles virus-infected stem cells produced by the method of the present invention show a viability of 80% is significantly increased to 80 hours or more, and thus the measles virus-infected stem cells may be administered in the form of an actual stem cell therapeutic agent. The present inventors named the anticancer stem cell therapeutic agent, obtained by infecting stem cells with the oncolytic virus, “ViroSTEM”.

In another aspect, the present invention is directed to a method for preventing or treating cancer comprising a step of administering the mesenchymal stem cells produced by the above method.

In another aspect, the present invention is directed to the use of the mesenchymal stem cells, produced by the above method, for the prevention or treatment of cancer.

In another aspect, the present invention is directed to the use of the mesenchymal stem cells, produced by the above method, for the production of a cell therapeutic agent for the prevention or treatment of cancer.

Hereinafter, the present invention will be described in more detail with reference to examples. These examples are only for illustrating the present invention in more detail, and it will be apparent to those of ordinary skill in the art that the scope of the present invention is not to be construed as being limited by these examples.

### EXAMPLE 1

### Isolation and Culture of Human Adipose Tissue-Derived Mesenchymal Stem Cells

Human adipose tissue obtained from abdominal fat by liposuction was isolated and washed with PBS. The tissue was minced and then digested using DMEM supplemented with collagenase type 1 (1 mg/ml) for 2 hours at 37° C. The tissue was washed with PBS and then centrifuged at 1,000 rpm for 5 minutes. The supernatant was suctioned and the pellet remaining on the bottom was washed with PBS and then centrifuged at 1,000 rpm for 5 minutes. The debris was removed by filtration through a 100 μm mesh, and the residue was washed with PBS, and then cultured in DMEM containing 10% FBS, 2 mM NAC and 0.2 mM ascorbic acid.

After allowing to stand overnight, non-adherent cells were washed with PBS and sub-cultured while replacing RKCM-N medium, that is, a keratinocyte-SFM medium (containing 5% FBS, 2 mM NAC, 0.2 mM ascorbic acid, 0.09 mM calcium, 5 ng/ml rEGF, 5 μg/ml insulin and 74 ng/ml hydrocortisone) every 2 days, thereby isolating adipose tissue-derived multipotent mesenchymal stem cells.

The adipose tissue-derived multipotent mesenchymal stem cells isolated as described above were stem cells cultured in the medium containing vitamin C, that is, mesenchymal stem cells pretreated with vitamin C.

### EXAMPLE 2

### Culture of Mesenchymal Stem Cells in Aspirin-Containing Medium

The stem cells cultured in the vitamin C-containing medium in Example 1 were seeded into 96-well cell culture plates at a density of 1×10⁴ cells/plate, and then cultured overnight for adhesion and stabilization. Next, the medium was replaced with a RKCM-N medium, that is, a keratinocyte-SFM medium (containing 5% FBS, 2 mM NAC, 0.2 mM ascorbic acid, 0.09 mM calcium, 5 ng/ml rEGF, 5 μg/ml insulin and 74 ng/ml hydrocortisone) supplemented with aspirin at a concentration of 0.5 mM, and then the cells were cultured for 24 hours.

Adipose tissue-derived mesenchymal stem cells obtained by culturing as described above were named Angel-Stem Cells (WO/2018/021879), and it could be confirmed through direct co-culture and indirect co-culture with cancer cells that the adipose tissue-derived mesenchymal stem cells had excellent anticancer effects.

### EXAMPLE 3

### Confirmation of Measles Virus Infection of Stem Cells

Whether the adipose-derived stem cells isolated in Example 1 or Example 2 would be infected with MVeGFP (GFP-tagged measles virus) was examined. Measles-GFP (MVeGFP) purchased from Limanis was amplified and quantified by calculating the titer, and an amplification method suitable for the culture field of the present invention and a titer determination method were established.

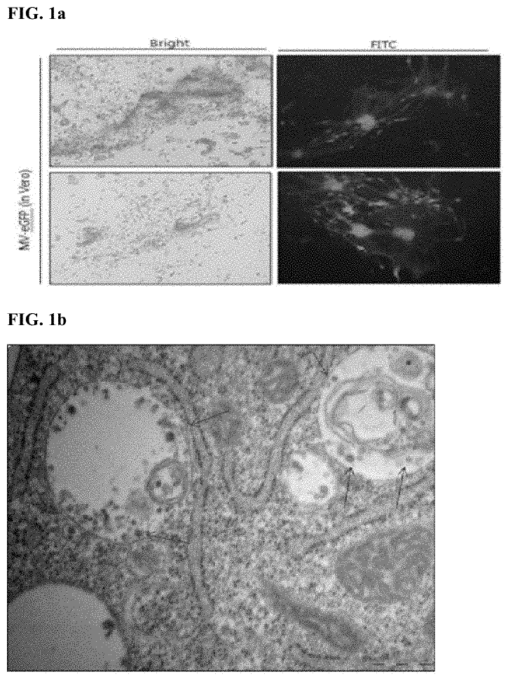

Specifically, the pelleted or flask-contained adipose-derived stem cells (2×10⁵ cells) were infected with measles vaccine virus (10⁶ TCID50) at 37° C. with gentle shaking every 30 minutes. Whether the stem cells obtained by culturing at 37° C. were infected with the measles vaccine virus was examined by fluorescence microscopy and electron microscopy (FIGS. 1a and 1b).

### EXAMPLE 4

### Evaluation of Killing Ability of Measles Virus in Cancer Cell Lines

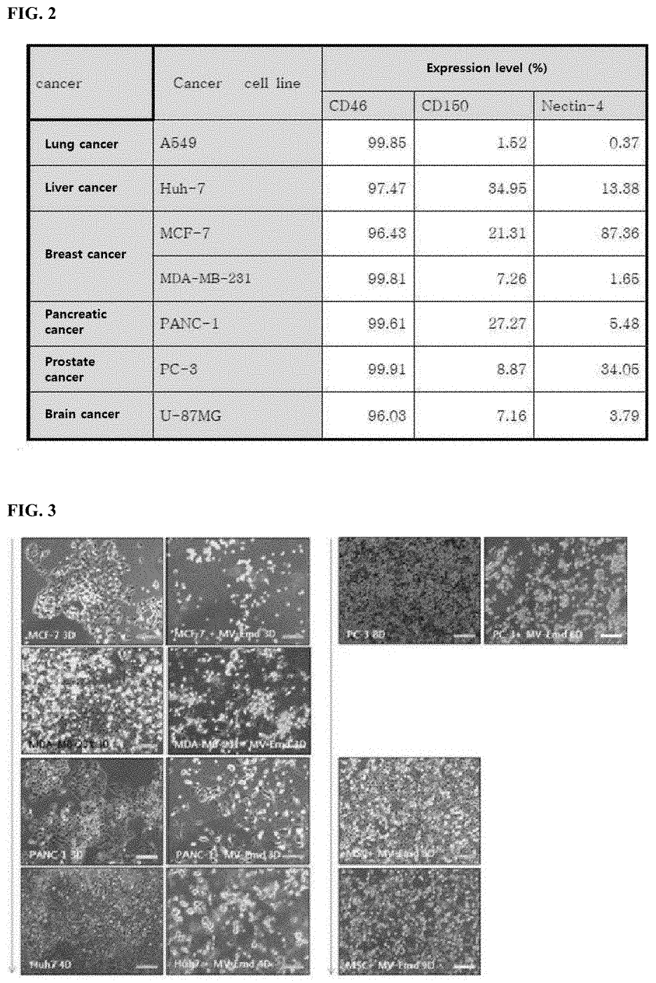

The killing ability of measles virus in various cancer cell lines can be measured by CD46, CD150, and nectin-4. Since expression of the three surface markers can indirectly indicate that the killing ability of the measles virus is increased, the surface markers in each cancer cell line were analyzed by FACS assay (FIG. 2).

Then, cancer cell death was analyzed by CPE (cytopathic effect) assay (FIG. 3). For CPE assay, cancer cells were plated on 24-well plates at a density of 2×10³ cells/well, and then infected with TCIE50 measles virus for 2 hours, followed by removal of the virus inoculum. 1 ml of cell culture medium was added to the cells which were then infected for 96 hours and washed twice with PBS. The remaining cells were fixed with 4% formaldehyde at room temperature for 10 minutes. After washing again with PBS, the cells were stained with 0.1% crystal violet (solubilized in 2% EtOH-DW), washed twice with DW, air-dried, and then photographed.

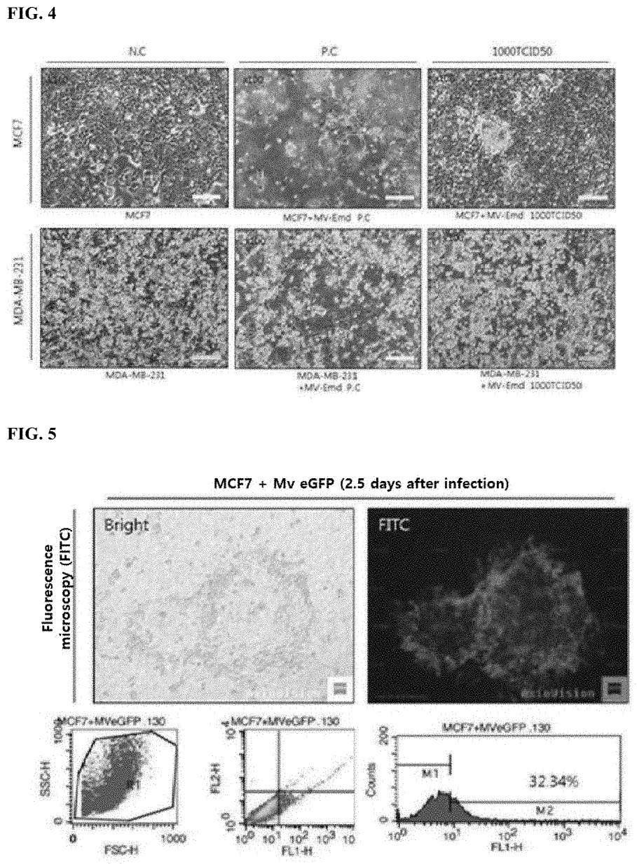

Cancer cell lines were infected with MVeGFP, and breast cancer cell lines were selected as cancer cell lines with a high probability of anticancer effect. The breast cancer killing ability of MVeGFP at 1000 TCID50/ml in the breast cancer cell lines (MCF-7: an estrogen-dependent cell line, and MDA-MB-231: an estrogen-independent cell line) was analyzed by CPE assay (FIG. 4). As a result, it was confirmed that, when CPE assay was performed at 1000 TCID50/ml, the positive control group showed CPE from day 2, and nearly 80% or more thereof formed CPE on day 3, and both MCF-7 and MDA-MB-231 started to form CPE on day 3.

In addition, breast cancer cells were infected with MVeGFP, and the extent of infection was quantitatively determined by FACS assay (FIG. 5).

### EXAMPLE 5

### Examination of Infection Specifications of ViroSTEM

The measles virus-introduced stem cells produced in Example 3 were named “ViroSTEM”. As the measles virus, measles vaccine virus was used or measles vaccine virus was used after further attenuation.

Based on the number of safe measles vaccine viruses and stem cells in humans (see the cell number and measles virus concentration by Mayo clinic), the infection specifications of ViroSTEM were established through testing for the infection environment, infection time and concentration (Table 1). In order to increase the extent of measles virus infection of stem cells, testing was performed while variously changing the infection environment.

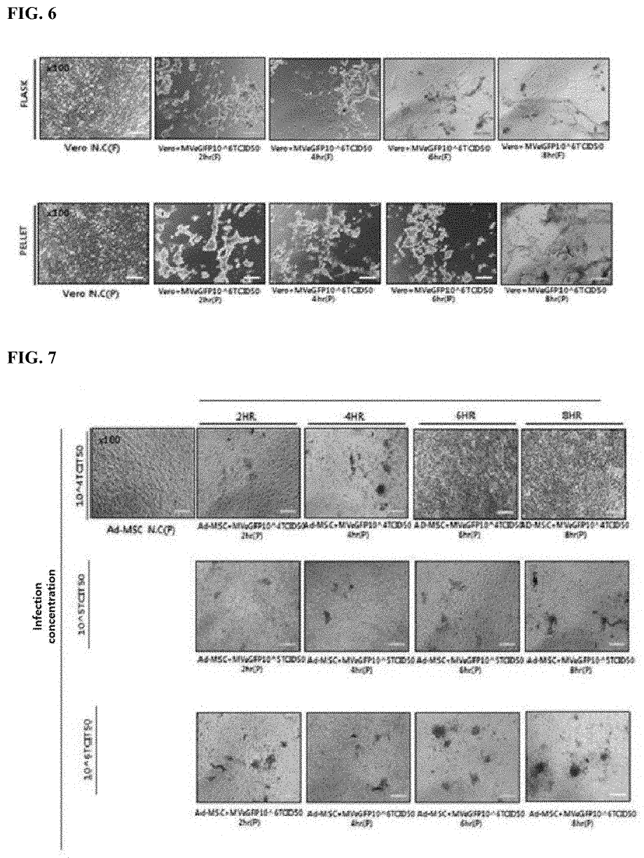

First, infection of flask-contained cells or pelleted cells was performed, and then the degree of CPE was examined. As a result, it was confirmed that the extent of infection was higher in the pelleted cell environment (FIG. 6), and the extent of infection could be increased by applying physical changes to the cells after infection.

Next, the infection time and concentration in the pelleted cell environment were changed, and then the degree of CPE was examined (FIG. 7). As a result, it could be seen that the extent of infection was the highest when the number of the mesenchymal stem cells was 2×10⁵ and the measles virus concentration was 1×10⁶ TCID50.

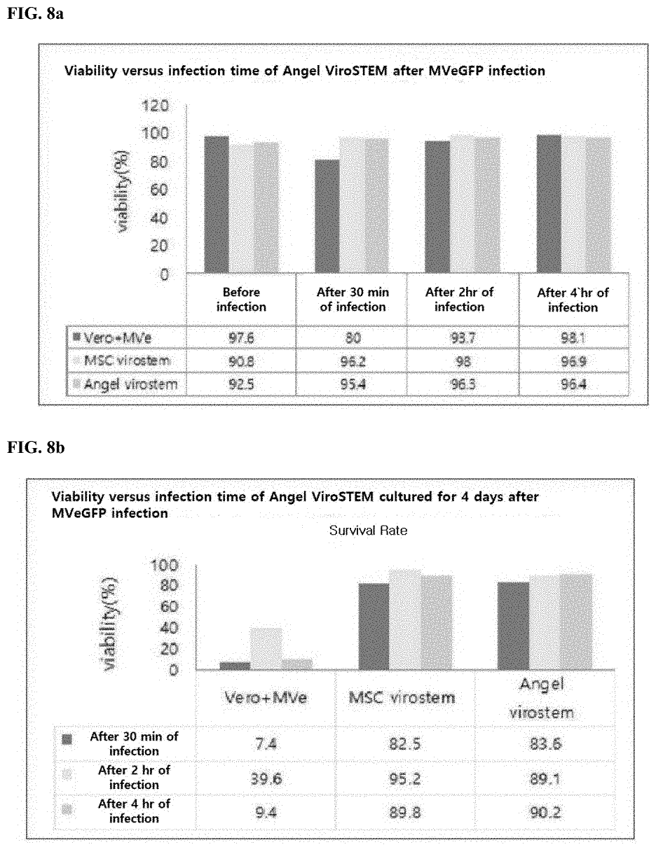

In addition, the infection time was changed and the viabilities of the stem cells infected with the measles virus for different times were analyzed immediately after infection and 4 days after infection. As a result, it was confirmed that an infection time of 30 minutes was most effective, and the extent of infection was excellent when the cells were not subjected to the culture step after infection with the measles virus (FIGS. 8a and 8b).

The infection specifications of ViroSTEM established in this way are shown in Table 1 below.

TABLE 1

| No. | Item | Specifications |
|---|---|---|
| 1 | Number of MSC cells | 2 × 10⁵ cells |
| 2 | Infection time | 30 minutes at 36 to 37° C. |
| 3 | Infection environment | Pelleted |
| 4 | MVeGFP concentration | 1 × 10⁶ TCID50 |

### EXAMPLE 6

### Increases in Viability and Stability During Cold Storage of Measles Virus-Infected Stem Cells by Aspirin Treatment

While ViroSTEM, which is mesenchymal stem cells into which measles virus has been introduced, was cold-stored in physiological saline containing aspirin and 30% autologous serum for 5 days, it was treated with aspirin at a concentration of 10 μg/ml to 100 μg/ml.

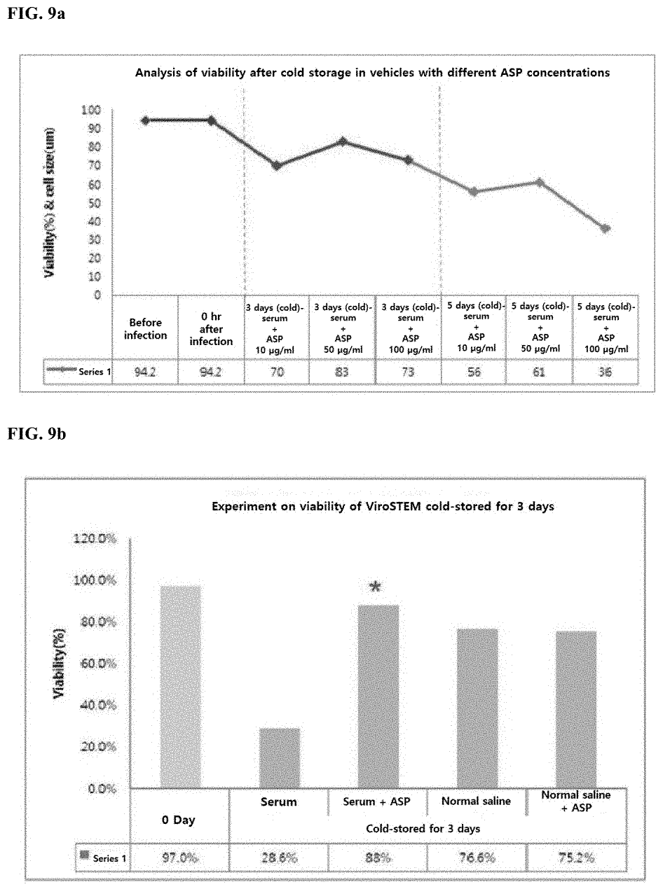

As a result, it was confirmed that the viability of the stem cells treated with aspirin significantly increased compared to that of the control group not treated with aspirin. The oncolytic virus-infected stem cells without any treatment showed a cell viability of about 28.6% when cold-stored in physiological saline containing 30% autologous serum for 3 days, but the cell viability of the anticancer stem cell therapeutic agent infected with measles virus increased to 88% or more when the stem cell therapeutic agent was treated with aspirin at a concentration of 50 μg/ml during cold storage in physiological saline containing 30% autologous serum (FIGS. 9a and 9b).

The period of time during which ViroSTEM cold-stored in physiological saline containing 50 μg/ml aspirin and 30% autologous serum was maintained as described above increased to 80 hours, indicating the stability of the virus in ViroSTEM cold-stored for 80 hours. After cold storage of ViroSTEM for 80 hours, the stem cells were cultured and then observed under a fluorescence microscope.

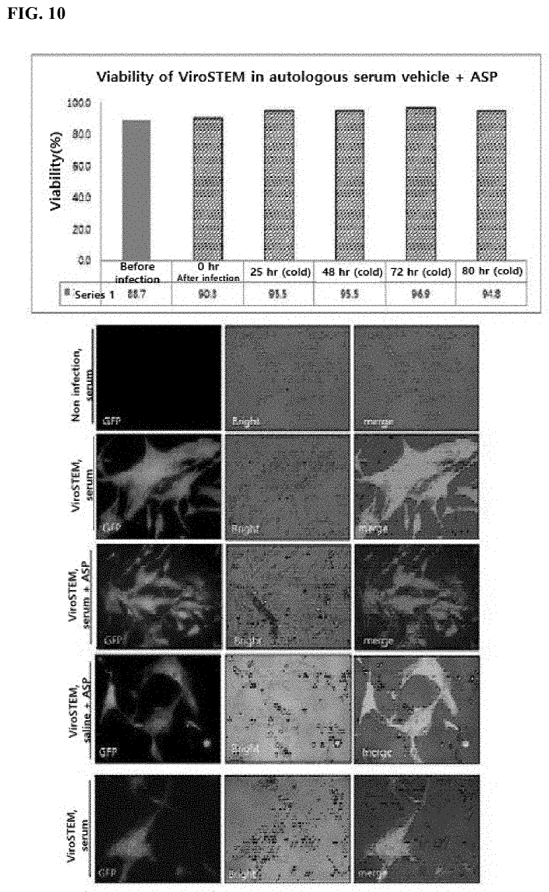

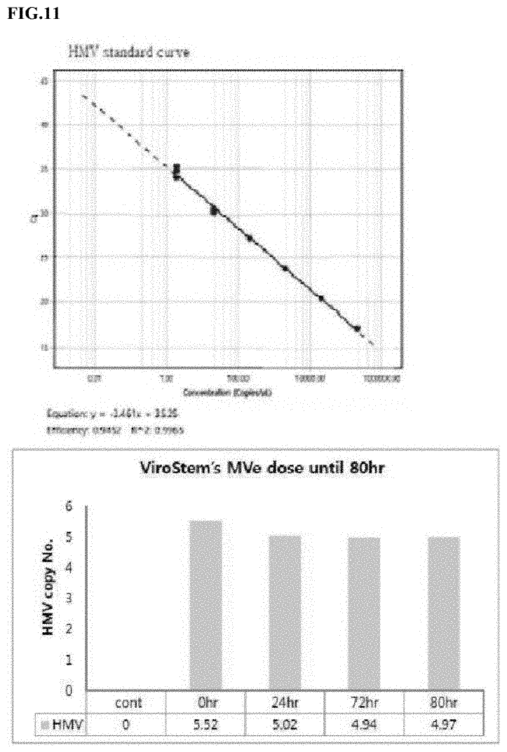

As a result, it could be confirmed that the GFP-tagged oncolytic virus was amplified and expressed up to 80 hours (FIG. 10).

Next, in order to test the stability of “ViroSTEM” obtained by infecting the anticancer stem cells (Angel-Stem Cells) with measles virus, the stem cells were infected with MVeGFP for 2 hours and then cold-stored in physiological saline containing 50 μg/ml aspirin and 30% autologous serum. How long the measles virus in ViroSTEM was maintained during cold storage was quantified by qPCR.

As a result, it could be confirmed that, when 50 μg/ml aspirin was contained in physiological saline containing 30% autologous serum, the measles vaccine in ViroSTEM was maintained for up to 80 hours during cold storage (FIG. 11).

### EXAMPLE 7

### Improvement in Anticancer Effect of Measles Virus-Infected Stem Cells by Aspirin Treatment

The anticancer effect of “ViroSTEM”, obtained by infecting the anticancer stem cells (Angel-Stem Cells obtained by aspirin treatment and culture) with measles virus, was evaluated.

“ViroSTEM” treated with aspirin was additionally seeded into cell culture dishes in which various cancer cells or cancer stem cells were adhered and cultured, respectively, and the anticancer effects of “ViroSTEM” on the cancer cells was evaluated. The metastatic breast cancer cell line (MDA-MB-231) inoculated with ViroSTEM treated with aspirin was stained using PKH67 Fluorescent Cell Linker Kits (SIGMA MIDI67).

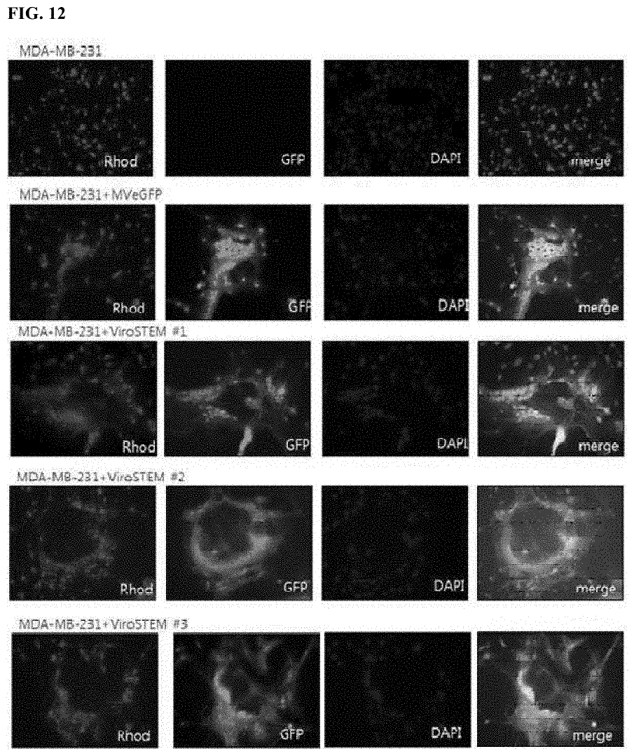

As a result, it could be confirmed that ViroSTEM treated with aspirin had an excellent anticancer effect against the metastatic breast cancer cell line (MDA-MB-231) (FIG. 12).

### INDUSTRIAL APPLICABILITY

According to the method for preparing oncolytic virus-containing mesenchymal stem cells and the method for storing oncolytic virus-containing mesenchymal stem cells according to the present invention, the infection efficiency of the oncolytic virus may be increased by aspirin treatment, the replication time of the virus may be prolonged, and lysis of the stem cells by the virus may be prevented, thereby improving the viability and survival period of the stem cells and preparing anticancer stem cells having excellent activity. The anticancer stem cell therapeutic agent produced in this way is maintained at high viability during cold storage due to aspirin treatment, and thus is very useful medically and industrially.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

1. A method for preparing oncolytic virus-containing mesenchymal stem cells having improved cell viability, the method comprising steps of:
(a) preparing pelleted mesenchymal stem cells;
(b) infecting the pelleted mesenchymal stem cells with an oncolytic virus; and
(c) separating the mesenchymal stem cells infected with the oncolytic virus to obtain mesenchymal stem cells into which the oncolytic virus has been introduced and which have improved cell viability.

2. The method of claim 1, wherein the mesenchymal stem cells in step (a) are cultured in a medium containing aspirin.

3. The method of claim 2, wherein the medium further contains vitamin C.

4. The method of claim 1, wherein the mesenchymal stem cells in step (a) are pretreated with vitamin C.

5. The method of claim 1, wherein the mesenchymal stem cells in step (b) are 1×10⁵ to 1×10⁶ cells.

6. The method of claim 1, wherein the mesenchymal stem cells are derived from a tissue selected from the group consisting of fat, uterus, bone marrow, muscle, placenta, umbilical cord blood, urine, hair follicle, and skin tissues.

7. The method of claim 1, wherein the oncolytic virus is measles virus.

8. The method of claim 1, wherein the oncolytic virus in step (b) has 1×10⁵ to 1×10⁷ TCID50.

9. The method of claim 1, wherein the oncolytic virus in step (b) is attenuated oncolytic virus.

10. The method of claim 1, wherein the infecting in step (b) is performed at 36 to 37° C. for 30 minutes.

11. The method of claim 1, wherein the oncolytic virus-infected mesenchymal stem cells in step (c) are additionally treated with aspirin.

12. The method of claim 11, wherein the treatment with aspirin is performed using a vehicle containing autologous serum.

13. The method of claim 12, wherein the treatment is performed at 0.1 to 10° C.

14. The method of claim 1, wherein the mesenchymal stem cells in step (c) are not further cultured after the oncolytic virus is introduced thereinto.

15. A method of storing mesenchymal stem cells, produced by the method of claim 1, using autologous serum containing aspirin.

16. The method of claim 15, wherein a concentration of the aspirin is 10 μg/ml to 100 μg/ml.

17. The method of claim 15, wherein a content of the autologous serum is 10% to 50%.

18. The method of claim 15, wherein the mesenchymal stem cells are stored at 0.1° C. to 10° C.

19. A cell therapeutic agent for cancer treatment containing, as an active ingredient, mesenchymal stem cells produced by the method of claim 1.

20. The cell therapeutic agent of claim 19, wherein the cancer is lung cancer, hematological tumor, ovarian cancer, myeloma, breast cancer, brain cancer, rectal cancer, colon cancer, colorectal adenocarcinoma, osteosarcoma, or cancer stem cells.

21. The cell therapeutic agent of claim 19, wherein the mesenchymal stem cells are derived from a tissue selected from the group consisting of fat, uterus, bone marrow, muscle, placenta, umbilical cord blood, urine, hair follicle, and skin tissues.
